(19) United States
(12) Patent Application Publication
(10) Pub. No.: US 2019/0001330 A1
(43) Pub. Date: Jan. 3, 2019

(54) CARTRIDGE FOR ENDOTOXIN DETECTION

(71) Applicants: Lonza Walkersville, Inc., Walkersville, MD (US); Lonza Cologne GmbH, Cologne (DE)

(72) Inventors: Gerald SANDO, Walkersville, MD (US); Candice STUMBAUGH, Walkersville, MD (US); Cindy HUNT, Walkersville, MD (US); Andreas HEINZE, Cologne (DE); Andy DURNAN, Cambridge (GB); Paul LEWIS, Cambridge (GB); James BAKER, Cambridge (GB); Will HARRIS, Cambridge (GB)

(21) Appl. No.: 16/020,552

(22) Filed: Jun. 27, 2018

Related U.S. Application Data
(60) Provisional application No. 62/525,864, filed on Jun. 28, 2017.

(51) Int. Cl. B01L 3/00; C12Q 1/37; G01N 21/78

(52) U.S. Cl. CPC B01L 3/502715; B01L 3/5023; B01L 3/50273; C12Q 1/37; G01N 2400/50; B01L 2200/026; B01L 2400/049; B01L 2200/16; G01N 2333/195; G01N 21/78

(57) ABSTRACT

The present invention is directed to methods, compositions and devices useful for the detection and/or quantification of a microbial contaminant, including an endotoxin. In embodiments, cartridges are provided that include dried compositions that are useful in absorbance-based assays and in combination with portable readers/devices.

### BACKGROUND OF THE INVENTION

### Field of the Invention

The present invention is directed to methods, compositions and devices useful for the detection and/or quantification of a microbial contaminant, including an endotoxin. In embodiments, cartridges are provided that include dried compositions that are useful in absorbance-based assays and in combination with portable readers/devices.

### Background of the Invention

Microbial contamination, including contamination from Gram positive bacteria, Gram negative bacteria, yeast, fungi, and molds, can cause severe illness and, in some cases, even death in humans. The pharmaceutical, medical device, and food industries often require frequent, accurate, and sensitive testing for the presence of such microbial contaminants to meet certain standards, for example, standards imposed by the United States Food and Drug Administration (USFDA) or Environmental Protection Agency.

What is needed is a convenient and rapid way to analyze samples for the presence of microbial contaminants, suitably with a compact, portable device that can be easily used in a variety of situations. The present invention meets these needs.

### BRIEF SUMMARY OF THE INVENTION

In embodiments, provided herein is a cartridge for determining the presence and/or amount of a microbial contaminant in a sample. In embodiments, the cartridge includes a housing including an optical sample well, a fluid inlet port and a conduit fluidly connecting the fluid inlet port and the optical sample well, a pump mechanism associated with the housing and fluidly connected to the fluid inlet port and the conduit, and a dried composition including a hemocyte lysate dried on the optical sample well.

In additional embodiments, the housing includes four optical sample wells, each comprising the dried composition including the hemocyte lysate and further including a chromogenic substrate dried on the optical sample wells, and wherein two of the four optical sample wells further include an agent representative of the microbial contaminant dried on the optical sample wells.

Suitably, the housing includes a liquid impermeable membrane fluidly connected to the optical sample well, and the housing can include a top section and a bottom section, mechanically connected to each other.

In embodiments, the pump mechanism is a two-position syringe, wherein a first position creates a vacuum to introduce the sample into the fluid inlet port and the conduit, and a second position provides transport from the conduit to the optical sample well.

Suitably, the hemocyte lysate is limulus amoebocyte lysate and the agent representative of the microbial contaminant is a bacterial endotoxin.

Also provided herein is a cartridge for determining the presence and/or amount of a microbial contaminant in a sample, the cartridge including a housing including a first optical sample well and a second optical sample well, a fluid inlet port and a conduit fluidly connecting the fluid inlet port and the first optical sample well and the second optical sample well, a two-position syringe attached to the housing and fluidly connected to the fluid inlet port and the conduit, wherein a first position creates a vacuum to introduce the sample into the fluid inlet port and the conduit, and a second position provides transport from the conduit to the first optical sample well and the second optical sample well, a dried composition including a hemocyte lysate and a chromogenic substrate dried on each of the first optical sample well and the second optical sample well, and an agent representative of the microbial contaminant dried on the second optical sample well.

Also provided are methods method for detecting the presence of a microbial contaminant in a sample, the method comprising introducing the sample into the fluid inlet port of the cartridges described herein and transferring the sample to the conduit, transferring the sample from the conduit to the optical sample well, and measuring an optical property of the sample in the optical sample well, wherein a change in the optical property is indicative of the presence of the microbial contaminant in the sample.

In embodiments of the methods, measuring the optical property is a change in absorbance of light at a preselected wavelength. Suitably, introducing the sample comprises creating a vacuum via a two-position syringe to introduce the sample into the fluid inlet port and the conduit. In further embodiments, the transferring comprises transporting via the two-position syringe the sample from the conduit to the optical sample wells, and wherein the transporting is stopped by a liquid impermeable membrane fluidly connected to the optical sample wells such that final volumes of the samples in each of the sample wells varies by less than about 10%.

Further embodiments, features, and advantages of the embodiments, as well as the structure and operation of the various embodiments, are described in detail below with reference to accompanying drawings.

### BRIEF DESCRIPTION OF THE DRAWINGS/FIGURES

FIGS. 1A-1B show a cartridge for endotoxin detection in accordance with an embodiment hereof.

FIG. 1C shows an optical sample well in accordance with an embodiment hereof.

FIG. 1D shows the introduction of a sample into a cartridge for endotoxin detection in accordance with an embodiment hereof.

FIG. 2 shows a liquid permeable membrane for use in a cartridge for endotoxin detection in accordance with an embodiment hereof.

FIG. 3 shows top and bottom sections of a cartridge for endotoxin detection in accordance with an embodiment hereof.

FIG. 4A shows a mounting apparatus for a cartridge for endotoxin detection in accordance with an embodiment hereof.

FIGS. 4B-4F show additional mounting apparatus for a cartridge for endotoxin detection in accordance with an embodiment hereof.

FIGS. 5A-5D show a further cartridge for endotoxin detection in accordance with an embodiment hereof.

FIGS. 6A-6B show reader devices in accordance with an embodiment hereof.

FIG. 6C shows an additional reader device in accordance with an embodiment hereof.

FIG. 7 shows components of a reader device in accordance with an embodiment hereof.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of ordinary skill in the art.

### Items of the Invention

1. A cartridge for determining the presence and/or amount of a microbial contaminant in a sample, the cartridge comprising:
   a. a housing including at least one optical sample well, a fluid inlet port and at least one conduit fluidly connecting the fluid inlet port and the at least one optical sample well;
   b. a pump mechanism associated with the housing and fluidly connected to the fluid inlet port and the at least one conduit; and
   c. a dried composition including a hemocyte lysate dried on the at least one optical sample well.
2. The cartridge of item 1 further comprising:
   a. a chromogenic substrate dried on the at least one optical sample well, and/or
   b. an agent representative of the microbial contaminant dried on the at least one optical sample well.
3. The cartridge of item 1 or 2, wherein the housing includes four optical sample wells, each comprising the dried composition including the hemocyte lysate and further including a chromogenic substrate dried on the optical sample wells, and wherein two of the four optical sample wells further include an agent representative of the microbial contaminant dried on the optical sample wells.
4. The cartridge according to any of items 1 to 3, wherein the housing includes at least one liquid impermeable membrane fluidly connected to the at least one optical sample well.
5. The cartridge according to any of items 1 to 4, wherein the housing includes a top section and a bottom section, mechanically connected to each other.
6. The cartridge according to any of items 1 to 5, wherein the pump mechanism is a two-position syringe, wherein a first position creates a vacuum to introduce the sample into the fluid inlet port and the at least one conduit, and a second position provides transport from the at least one conduit to the at least one optical sample well.
7. The cartridge according to any of items 1 to 6, wherein the hemocyte lysate is limulus amoebocyte lysate (LAL).
8. The cartridge of item 7, wherein the LAL is LAL substantially free of coagulogen.
9. The cartridge of item 7, wherein the LAL is clarified LAL.
10. The cartridge according to any of items 1 to 6, wherein the hemocyte lysate is Tachypleus amoebocyte lysate or Carcinoscorpius amoebocyte lysate.
11. The cartridge according to any of items 2 to 10, wherein the agent representative of the microbial contaminant is a bacterial endotoxin.
12. The cartridge according to any of items 1 to 11 wherein the dried composition comprises about 1 μg to about 50 μg hemocyte lysate, and optionally about 0.1 μg to 5 μg chromogenic substrate.
13. A cartridge for determining the presence and/or amount of a microbial contaminant in a sample, the cartridge comprising:
   a. a housing including at least a first optical sample well and a second optical sample well, a fluid inlet port and at least one conduit fluidly connecting the fluid inlet port and the first optical sample well and the second optical sample well;
   b. a two-position syringe attached to the housing and fluidly connected to the fluid inlet port and the at least one conduit, wherein a first position creates a vacuum to introduce the sample into the fluid inlet port and the conduit, and a second position provides transport from the conduit to the first optical sample well and the second optical sample well;
   c. a dried composition including a hemocyte lysate and a chromogenic substrate dried on each of the first optical sample well and the second optical sample well; and
   d. an agent representative of the microbial contaminant dried on the second optical sample well.
14. The cartridge of item 13, wherein the housing includes four optical sample wells, each comprising the dried composition dried on the optical sample wells, and wherein two of the four optical sample wells include the agent representative of the microbial contaminant dried on the optical sample wells.
15. The cartridge of item 13 or 14, wherein the housing includes at least one liquid impermeable membrane fluidly connected to the optical sample wells.
16. The cartridge according to any of items 13 to 15, wherein the housing includes a top and a bottom section, mechanically connected to each other.
17. The cartridge according to any of items 13 to 16, wherein the hemocyte lysate is limulus amoebocyte lysate (LAL).
18. The cartridge of item 17, wherein the wherein the LAL is LAL substantially free of coagulogen.
19. The cartridge of item 17, wherein the wherein the LAL is clarified LAL.
20. The cartridge according to any of items 13 to 16, wherein the hemocyte lysate is Tachypleus amoebocyte lysate or Carcinoscorpius amoebocyte lysate.
21. The cartridge according to any of items 1 to 20, wherein the agent representative of the microbial contaminant is a bacterial endotoxin.
22. The cartridge according to any of items 13 to 21 wherein the dried composition comprises about 1 μg to about 50 μg hemocyte lysate, and about 0.1 μg to 5 μg chromogenic substrate.
23. A method for detecting the presence of a microbial contaminant in a sample, the method comprising:
   a. introducing the sample into the fluid inlet port of the cartridge of item 1 to 12 and transferring the sample to the conduit;
   b. transferring the sample from the at least one conduit to the at least one optical sample well; and
   c. measuring an optical property of the sample in the at least one optical sample well, wherein a change in the optical property is indicative of the presence of the microbial contaminant in the sample.
24. The method of item 23, wherein the measuring the optical property is a change in absorbance of light at a preselected wavelength.
25. The method of item 24, wherein the change in absorbance of light at a preselected wavelength is compared to a standard curve.
26. The method of item 25, wherein the standard curve is an archived standard curve.
27. The method according to any of items 16 to 22, wherein the introducing the sample comprises creating a vacuum via the pump mechanism to introduce the sample into the fluid inlet port and the at least one conduit.
28. The method according to any of items 23 to 27, wherein the transferring comprises transporting via the pump mechanism the sample from the at least one conduit to the at least one optical sample well, and wherein the transporting is stopped by at least one liquid impermeable membrane fluidly connected to the at least one optical sample well.
29. A method for detecting the presence of a microbial contaminant in a sample, the method comprising:
   a. introducing the sample into the fluid inlet port of the cartridge of item 13 to 22 and transferring the sample to the at least one conduit;
   b. transferring the sample from the conduit to the optical sample wells; and
   c. measuring an optical property of the sample in the optical sample wells, wherein a change in the optical property is indicative of the presence of the microbial contaminant in the sample.
30. The method of item 29, wherein the measuring the optical property is a change in absorbance of light at a preselected wavelength.
31. The method of item 30, wherein the change in absorbance of light at a preselected wavelength is compared to a standard curve.
32. The method of item 31, wherein the standard curve is an archived standard curve.
33. The method according to any of items 29 to 32, wherein the introducing the sample comprises creating a vacuum via the two-position syringe to introduce the sample into the fluid inlet port and the conduit.
34. The method of according to any of items 29 to 33, wherein the transferring comprises transporting via the two-position syringe the sample from the conduit to the optical sample wells, and wherein the transporting is stopped by a liquid impermeable membrane fluidly connected to the optical sample wells such that final volumes of the samples in each of the sample wells varies by less than about 10%.

### Testing for Microbial Contaminants

A variety of assays have been developed to detect the presence and/or amount of a microbial contaminant in a test sample. Hemocyte lysates prepared from the hemolymph of crustaceans, for example, horseshoe crabs, are often utilized. These assays typically exploit, in one way or another, a clotting cascade that occurs when the hemocyte lysate is exposed to a microbial contaminant. Examples of hemocyte lysates include the amoebocyte lysate (AL) produced from the hemolymph of a horseshoe crab, Limulus polyphemus, Tachypleus gigas, Tachypleus tridentatus, and Carcinoscorpius rotundicauda. Amoebocyte lysates produced from the hemolymph of Limulus, Tachypleus, and Carcinoscorpius species are referred to as Limulus amoebocyte lysate (LAL), Tachypleus amoebocyte lysate (TAL), and Carcinoscorpius amoebocyte lysate (CAL), respectively.

Assays that use LAL include, for example, gel clot assays, end point turbidimetric assays, kinetic turbidimetric assays, and endpoint chromogenic assays (Prior (1990) “Clinical Applications of the Limulus Amoebocyte Lysate Test” CRC PRESS 28-34). These assays, however, suffer from one or more disadvantages including reagent expense, assay speed and limited sensitivity ranges. Also, these assays typically require that samples be sent to a testing facility removed from the origin of the sample being tested.

### Cartridge for Endotoxin Detection

In embodiments, provided herein is a cartridge for determining the presence and/or amount of a microbial contaminant in a sample.

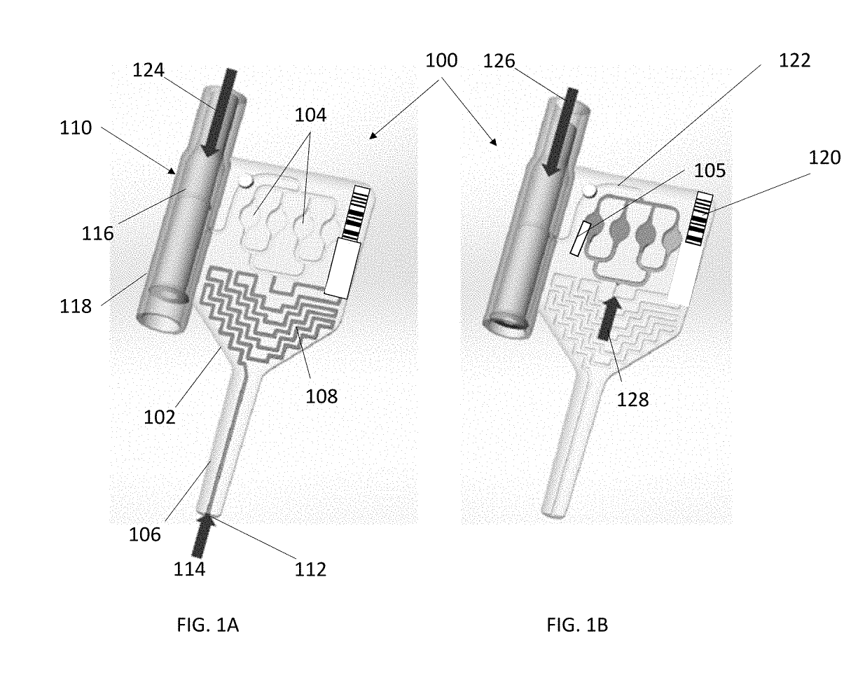

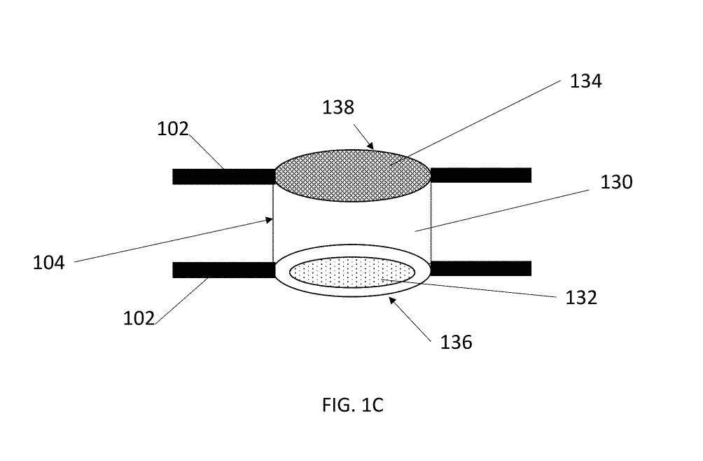

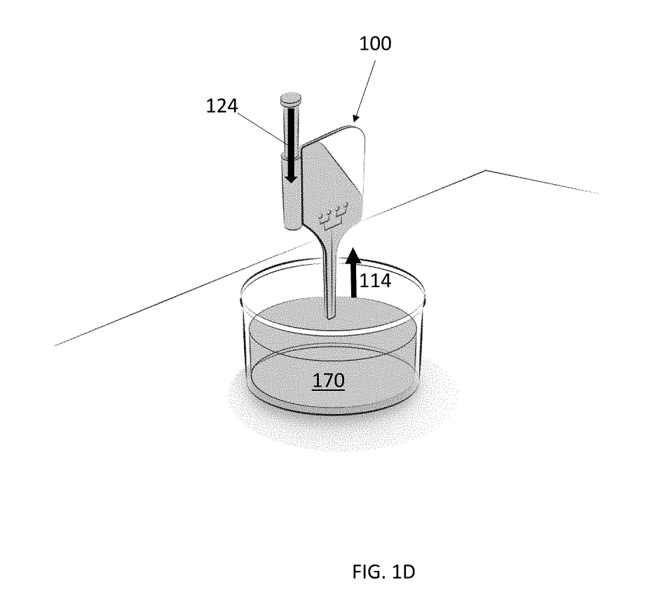

As shown in FIG. 1A, in embodiments, cartridge 100, includes housing 102, which provides a structural support for the cartridge. As used herein “cartridge” means a self-contained element for receiving and holding a sample that is to be tested for the presence and/or amount of a microbial contaminant.

Housing 102 can be prepared from any suitable material including various plastics or glasses. Housing 102 includes optical sample well 104. FIG. 1C shows a side view of optical sample well 104, illustrating well wall 130, which creates a vessel or ampule between the upper and lower surfaces of housing 102. Optical sample well 104 is suitably constructed from a plastic or glass material, and suitably is optically clear or transparent, so as to allow light to pass through the bottom and top of the optical sample well, thereby allowing the light to contact a sample contained within the optical sample well. In embodiments, optical sample well 104, as well as the remainder of housing 102, can be prepared from a yellow colored glass or polymer, which suitably blocks light of about 400 nm to about 450 nm from passing (i.e., 405 nm), but allows other wavelengths to pass. Such embodiments reduce the amount light passing through optical sample well 104, reducing cross-talk and increasing the sensitivity of the methods described herein by reducing cross-talk and stray light.

Housing 102 of cartridge 100 also further includes fluid inlet port 106 and conduit 108. Fluid inlet port 106 is suitably a long or slender section of housing 102, inside of which conduit 108 is housed, and which has an opening at tip 112, suitably for contacting a sample 112. Fluid inlet port 106 is designed so as to allow a sample to be drawn upward, in sample introduction direction 114, into the fluid inlet port. Conduit 108 fluidly connects fluid inlet port 106 and optical sample well 104. That is, conduit 108 provides a tubular or microfluidic connection between tip 112 of fluid inlet port 106 and optical sample well 104, that allows for transfer of a liquid sample between the fluid inlet port, after being drawn into the port, through conduit 108, and into optical sample well 104. In embodiments, conduit 108 can be formed as channels cut into the surface of housing 102, or can be formed as tubing or microtubing from various polymers such as poly(styrene), etc.

As shown in FIG. 1A, upon introduction of a sample into fluid inlet port 106, the sample travels to an initial position within conduit 108, where the sample is held and maintained prior to transferring to optical sample well 104.

In embodiments, cartridge 100 further includes pump mechanism 110 associated with housing 102. Pump mechanism 110 suitably is a two-position syringe, which includes barrel 118 and plunger 116, which can slide within the barrel. Pump mechanism 110 can be attached directly to housing 102 via a suitable mechanism (e.g., glue, adhesive, mechanical bands, wraps or staples, etc.) or can be an element prepared as an integrated part of housing 102 (for example the top section of the housing). Pump mechanism 110 is fluidly connected to fluid inlet port 106 and conduit 108, such that upon actuation of pump mechanism 110, a sample can be drawn into fluid inlet port 106, and then into conduit 108, for example as shown in FIG. 1A, with 114 illustrating the sample introduction direction.

FIG. 1A shows the actuation of pump mechanism 110, suitably a two-position syringe in first position 124, which causes the sample to transfer into fluid inlet port 106 and conduit 108, but to remain in conduit 108 and not pass further into optical sample well 104. For example, a vacuum can be generated by two position syringe to introduce or draw the sample through tip 112, into fluid inlet port 106, and then into conduit 108. This vacuum can suitably be generated by vacuum conduit 122 (FIG. 1B), positioned downstream from optical sample well 104. However, in other embodiments, a vacuum can be generated between conduit 108 and sample well 104, which allows for the initial introduction of the sample into the conduit.

FIG. 1B shows the actuation of pump mechanism 110 in second position 126, which provides transport of the sample from conduit 108 to optical sample well 104. In embodiments, second position 126 can cause a further vacuum, for example generated by vacuum conduit 122, to draw the sample from conduit 108 into optical sample well 104, via sample flow direction 128.

Introducing or drawing a sample into fluid inlet port 106, and then conduit 108, and maintaining the sample in conduit 108 with pump mechanism 110 at first position 124, as shown in FIG. 1A, allows cartridge 100 with the sample to be prepared at a location of sampling (e.g., an assembly line, plant, facility, sample vats or storage tanks), and then maintained in a state of readiness, prior to detection or measurement. An advantage of this design is that the sample can be held in cartridge 100 for a period of time (suitably minutes (e.g., 10-30 minutes) up to about 1-2 hours, or longer) prior to making a measurement of the sample, thus allowing for multiple different samples to be taken without the fear of loss of sample quality. In addition, if further activities are required following the taking of a sample, these can be carried out, and then the sample analyzed at a later time.

Pump mechanism 110, suitably a two-position syringe as described herein, can be primed (i.e., pulled out to create a vacuum source) as shown in FIG. 1D, so as to generate a vacuum that upon actuation of plunger 116 to first position 124, introduces sample 170 into fluid inlet port 106 and conduit 108, by drawing sample 170 up into sample inlet port 106 (see FIG. 1A), for example, via vacuum conduit 122. Upon actuation of plunger 116 to second position 126 (see FIG. 1B), sample 170 is transported to optical sample well 400, suitably by the action of a vacuum via vacuum conduit 122.

In embodiments, the length of conduit 108, which can include the length from the tip 112 of fluid inlet port 106, to end of the location of the sample in first position 124, is on the order of about 1 cm to about 5 cm, more suitably about 1 cm to about 3 cm. Conduit 108 can include individual channels or sections that are on the order of about 0.2 mm to about 1.5 mm in length, connected to a central source point, which make up the full length of conduit 108. In additional embodiments, conduit 108 can be a continuous single channel extending from tip 112 of fluid inlet port 106, to end of the location of the sample in first position 124. For example, the length of the sections of the conduit, or the full length of the conduit, is on the order of about 0.2 mm to about 1.0 mm, about 0.2 mm to about 0.8 mm, about 0.2 mm to about 0.7 mm, about 0.4 mm to about 0.6 mm, or about 0.3 mm about 0.4 mm, about 0.5 mm, about 0.6 mm, about 0.7 mm, about 0.8 mm, about 0.9 mm or about 1.0 mm. The diameter or cross-sectional width of conduit 108 is suitably on the order of about 0.1 mm to about 1 mm, about 0.5 mm to about 1 mm, about 0.5 mm to about 0.8 mm, or about 0.1 mm, about 0.2 mm, about 0.3, about 0.4 mm, about 0.5 mm, about 0.6 mm, about 0.7 mm or about 0.8 mm. Use of a cross-sectional width of conduit 108 on the order of about 0.5 mm helps in reducing wicking and preventing sample loss. In embodiments, as shown in FIG. 1A, conduit 108 can include a bent or doubled-over pattern (or other similar orientation that maximizes length of conduit 108 while minimizing surface area), and in other embodiments, conduit 108 can be a substantially straight channel from tip 112 to end of the location of the sample in first position 124. In other embodiments, more than one conduits can be present such as 2, 3, 4, 5, 6, 7, 8, 9, or 10 or more conduits.

The use of sections of conduit 108 having lengths on the order of about 0.2 mm to about 1 mm helps in reducing wicking, air pocket formation and loss of sample, while the sample is being readied for analysis, including in the devices/readers described herein.

Sample sizes that can be maintained within conduit 108, prior to measurement and analysis are suitably on the order of about 25 μl to about 200 μl, more suitably about 50 μl to about 150 μl, about 50 μl to about 100 μl, about 50 μl to about 80 μl, or about 40 μl, about 50 μl, about 60 μl, about 65 μl, about 70 μl, about 75 μl, about 80 μl, about 90 μl, or about 100 μl.

In embodiments, optical sample well 104 includes dried composition 132 on one or more surfaces of the sample well. In embodiments, as shown in FIG. 1C, dried composition 132 can be dried onto bottom 136 of optical sample well 104, though in other embodiments, the dried composition can be dried on top 138, well wall (side) 130, and/or bottom 136, of optical sample well 104.

The size of optical sample well 104, and thus suitably the volume that can be maintained with the well, are dictated by the height of well wall 130, and the diameter of the top 136 and bottom 138 of the well. Suitably, optical sample well 104 will have a height on the order of about 100 μm to about 20 mm, more suitably about 100 μm to about 10 mm or about 100 μm to about 5 mm, and a diameter on the order of about 100 μm to about 20 mm, more suitably about 100 μm to about 10 mm or about 100 μm to about 5 mm. In embodiments, optical sample well 104 suitably holds about 1 μL to about 5 mL of sample, or about 1 μL to about 1 mL or about 1 μL to about 500 μL, about 1 μL to about 50 μL, about 1 μL to about 20 μL, about 1 μL to about 10 μL, or about 1 μl, about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, or about 15 μl.

In embodiments, dried composition 132 includes a hemocyte lysate, dried onto the optical sample well 104. As used herein “dried composition” includes substances that are freeze-dried, lyophilized or vitrified, to form a dried cake, powder, crystal or film on the surface of optical well 104. Methods of lyophilization or vitrification are known in the art. In exemplary embodiments, the amount of the dried composition in each optical sample well is the same, or within about 1-30% variation, suitably less than 10% or less than 5% variation, of each other, to provide a consistent amount of dried composition and thus re-hydrated composition, as described herein.

As used herein, the term, “hemocyte lysate” means any lysate, or a fraction or component thereof, produced by the lysis and/or membrane permeabilization of hemocytes, for example, amoebocytes and hemolymph cells, (i) extracted from a crustacean or insect and/or (ii) cultured in vitro after extraction from the host. Hemocyte cellular material that has been extruded from hemolymph cells by contact with a membrane permeabilization agent such as a Ca²⁺ ionophore or the like (i.e., extruded other than by lysis) or otherwise extracted without cellular lysis is also considered to be a hemocyte lysate.

An exemplary hemocyte lysate is an amoebocyte lysate prepared from the blood of a crustacean, for example, a horseshoe crab or Jonah crab. As used herein, the term “amoebocyte lysate” is understood to mean any lysate or fraction or component thereof produced by the lysis, extrusion, or extraction of the cellular contents from amoebocytes extracted from a crustacean, for example, a horseshoe crab. The amoebocyte lysate comprises at least one component of an enzymatic cascade and/or produces a clot in the presence of an endotoxin, for example, a Gram negative bacterial endotoxin and/or a glucan, for example, a (1→3)-β-D glucan, produced by a yeast or a mold. Exemplary amoebocyte lysates can be derived from horseshoe crabs, which include crabs belonging to the Limulus genus, for example, Limulus polyphemus, the Tachypleus genus, for example, Tachypleus gigas, and Tachypleus tridentatus, and the Carcinoscorpius genus, for example, Carcinoscorpius rotundicauda.

Limulus amoebocyte lysate (LAL) is employed as the amoebocyte lysate of choice in many bacterial endotoxin assays because of its sensitivity, specificity, and relative ease for avoiding interference by other components that may be present in a sample. LAL, when combined with a sample containing a bacterial endotoxin and optionally with certain LAL substrates, reacts with the endotoxin in the sample to produce a detectable product, such as a gel, an increase in turbidity, or a colored or light-emitting product, in the case of a synthetic chromogenic substrate. The product may be detected, for example, either visually or by the use of an optical detector.

When bacterial endotoxin is contacted with LAL, the endotoxin initiates a series of enzymatic reactions, referred to in the art as the Factor C pathway, that can involve three serine protease zymogens called Factor C, Factor B, and pro-clotting enzyme. Upon exposure to endotoxin, the endotoxin-sensitive factor, Factor C, is activated. Activated Factor C thereafter hydrolyses and activates Factor B, whereupon activated Factor B activates proclotting enzyme to produce a clotting enzyme. The clotting enzyme thereafter hydrolyzes specific sites, for example, Arg₁₈-Thr₁₉ and Arg₄₆-Gly₄₇ of coagulogen, an invertebrate, fibrinogen-like clottable protein, to produce a coagulin gel. See, for example, U.S. Pat. No. 5,605,806.

Methods for enhancing the sensitivity of a hemocyte lysate for endotoxin, for example, include, without limitation, aging the crude hemocyte lysate, adjusting pH, adjusting the concentration of divalent cations, adjusting the concentration of coagulogen, chloroform extraction, and the addition of serum albumin, biocompatible buffers and/or biological detergents.

For example, in embodiments the hemocyte lysate for use in the dried compositions described herein can be a hemocyte lysate that is substantially free of coagulogen. In another embodiment, the hemocyte lysate that is substantially free of coagulogen is LAL substantially free of coagulogen. One of skill in the art, upon reading the present disclosure, would appreciate that a reduction in various amounts of coagulogen will result in increasing levels of speed, sensitivity and/or separation in a chromogenic assay, e.g., an LAL assay. In some embodiments, the term “substantially free” refers to hemocyte lysate having less than 50%, less than 40%, less than 30%, less than 20%, less than 10%, less than 5%, less than 2%, less than 1% or less than 0.5% (wt/wt) of coagulogen relative to total protein in the hemocyte lysate as measured by SDS-PAGE with protein stain and confirmed by Western blot. In some embodiments, the term “substantially free” refers to LAL having less than 50%, less than 40%, less than 30%, less than 20%, less than 10%, less than 5%, less than 2%, less than 1% or less than 0.5% (wt/wt) of coagulogen relative to total protein in the LAL as measured by SDS-PAGE with protein stain and confirmed by Western blot. In some embodiments, the term “substantially free” refers to clarified LAL having less than 50%, less than 40%, less than 30%, less than 20%, less than 10%, less than 5%, less than 2%, less than 1% or less than 0.5% (wt/wt) of coagulogen relative to total protein in the LAL as measured by SDS-PAGE with protein stain and confirmed by Western blot.

In some embodiments, the term “substantially free” refers to hemocyte lysate having less than 10% or less than 5% (wt/wt) of coagulogen relative to total protein in the hemocyte lysate as measured by SDS-PAGE with protein stain and confirmed by Western blot. In some embodiments, the term “substantially free” refers to LAL having less than 10% or less than 5% (wt/wt) of coagulogen relative to total protein in the LAL as measured by SDS-PAGE with protein stain and confirmed by Western blot. In some embodiments, the term “substantially free” refers to clarified LAL having less than 10% or less than 5% (wt/wt) of coagulogen relative to total protein in the LAL as measured by SDS-PAGE with protein stain and confirmed by Western blot.

In some embodiments, the term “substantially free” refers to hemocyte lysate having a concentration of coagulogen at less than about 20 μg/μL, less than about 15 μg/μL, less than about 10 μg/μL, less than about 5 μg/μL, less than about 4 μg/μL, less than about 3 μg/μL, less than about 2 μg/μL, or less than about 1 μg/μL. In some embodiments, the term “substantially free” refers to LAL having a concentration of coagulogen at less than about 20 μg/μL, less than about 15 μg/μL, less than about 10 μg/μL, less than about 5 μg/μL, less than about 4 μg/μL, less than about 3 μg/μL, less than about 2 μg/μL, or less than about 1 μg/μL. In some embodiments, the term “substantially free” refers to clarified LAL having a concentration of coagulogen at less than about 20 μg/μL, less than about 15 μg/μL, less than about 10 μg/μL, less than about 5 μg/μL, less than about 4 μg/μL, less than about 3 μg/μL, less than about 2 μg/μL, or less than about 1 μg/μL.

In some embodiments, the term “substantially free” refers to hemocyte lysate having a concentration of coagulogen of 20 μg/μL to 0.001 μg/μL, 15 μg/μL to 0.01 μg/μL, 10 μg/μL to 0.1 μg/μL, 5 μg/μL to 0.5 μg/μL, 4 μg/μL to 0.5 μg/μL, 3 μg/μL to 0.5 μg/μL, 2 μg/μL to 0.5 μg/μL, or less than 1 μg/μL. In some embodiments, the term “substantially free” refers to LAL having a concentration of coagulogen of 20 μg/μL to 0.001 μg/μL, 15 μg/μL to 0.01 μg/μL, 10 μg/μL to 0.1 μg/μL, 5 μg/μL to 0.5 μg/μL, 4 μg/μL to 0.5 μg/μL, 3 μg/μL to 0.5 μg/μL, 2 μg/μL to 0.5 μg/μL, or less than 1 μg/μL. In some embodiments, the term “substantially free” refers to clarified LAL having a concentration of coagulogen of 20 μg/μL to 0.001 μg/μL, 15 μg/μL to 0.01 μg/μL, 10 μg/μL to 0.1 μg/μL, 5 μg/μL to 0.5 μg/μL, 4 μg/μL to 0.5 μg/μL, 3 μg/μL to 0.5 μg/μL, 2 μg/μL to 0.5 μg/μL, or less than 1 μg/μL.

In some embodiments, the term “substantially free” refers to hemocyte lysate having a concentration of coagulogen of 10 μg/μL to 1 μg/μL, 5 μg/μL to 1 μg/μL, 4 μg/μL to 1 μg/μL, 3 μg/μL to 1 μg/μL, 2 μg/μL to 1 μg/μL, or less than 1 μg/μL. In some embodiments, the term “substantially free” refers to LAL having a concentration of coagulogen of 10 μg/μL to 1 μg/μL, 5 μg/μL to 1 μg/μL, 4 μg/μL to 1 μg/μL, 3 μg/μL to 1 μg/μL, 2 μg/μL to 1 μg/μL, or less than 1 μg/μL. In some embodiments, the term “substantially free” refers to clarified LAL having a concentration of coagulogen of 10 μg/μL to 1 μg/μL, 5 μg/μL to 1 μg/μL, 4 μg/μL to 1 μg/μL, 3 μg/μL to 1 μg/μL, 2 μg/μL to 1 μg/μL, or less than 1 μg/μL. The concentration of coagulogen may be determined, e.g., using absorbance spectroscopy, quantification of an SDS-PAGE gel band or Western blot band, or any other method known to measure coagulogen concentration. In some embodiments, the measured concentration of coagulogen in the “hemocyte lyate substantially free of coagulogen” or the “LAL substantially free of coagulogen” or “clarified LAL” cannot be precisely determined as it is within the margin of error of the minimum detection amount using conventional detection methods. Exemplary LAL substantially free of coagulogen is described in U.S. patent application Ser. No. 15/868,318, filed Jan. 11, 2018, and U.S. patent application Ser. No. 15/668,101, filed Aug. 3, 2017, the disclosures of both which are incorporated by reference herein in their entirety.

One of skill in the art can appreciate that different methods may be used to remove the coagulogen from the hemocyte lystate, e.g., LAL. Each of these methods, may differ in efficiency, rate of purification, cost, and effort, but are within the knowledge of the skilled artisan. In some embodiments, the hemocyte lystate, e.g., LAL, is substantially free of coagulogen, wherein the composition is made by a method comprising: (a) obtaining a solution derived from lysed amebocytes from Limulus polyphemus; (b) combining the solution from (a) with a buffer; (c) subjecting the combination from (b) to continuous tangential flow filtration (TFF) using a 20 kDa to 50 kDa membrane filter to produce a retentate; and (d) centrifuging the retentate from (c) at greater than 20,000×g for greater than 25 minutes to produce a supernatant, wherein the supernatant is clarified LAL that is substantially free of coagulogen.

In some embodiments, the hemocyte lystate, e.g., LAL, can be made using tangential flow filtration. Tangential flow filtration (TFF) refers to cross-flow filtration wherein the majority of the feed flow travels tangentially across the surface of the filter, rather than into the filter. By using TFF, the retentate comprising the majority of LAL proteins (which can foul the filter) is substantially washed away during the filtration process, and coagulogen is filtered into the permeate. In some embodiments, the TFF is a continuous process, i.e., continuous tangential flow filtration or continuous TFF, unlike batch-wise dead-end filtration. In some embodiments, continuous TFF comprises adding a diafiltration solution, i.e., water or buffer, to the sample at the same rate that permeate is generated, and thus the sample volume remains constant while the components that can freely permeate the filter are washed away. In some embodiments, diafiltration is a type of tangential flow filtration. Diafiltration refers to the fractionation process that washes smaller molecules through a membrane or filter and leaves larger molecules in the retentate without ultimately changing volume. A diafiltration volume, or DV, is the volume of sample before the diafiltration solution is added. In embodiments, using more diafiltration volumes in tangential flow filtration results in greater removal of permeate.

In some embodiments, the hemocyte lysate is a clarified limulus amebocyte lyate. The term “clarified limulus amebocyte lysate” (or “clarified LAL”) that is substantially free of coagulogen refers to LAL substantially free of coagulogen, discussed above, that has been further treated to remove components that create a cloudy appearance of the LAL. In embodiments, clarified LAL is created by centrifuging LAL substantially free of coagulogen. In some embodiments, the term “clarified LAL” refers to LAL that has been centrifuged at greater than 1800 g (i.e., 1800×gravity), greater than 2200 g, greater than 2600 g, greater than 3000 g, greater than 3400 g, greater than 3800 g, greater than 4200 g, greater than 4600 g, greater than 5000 g, greater than 5400 g, greater than 5800 g, greater than 6000 g, greater than 6100 g, or greater than 6200 g for a period of time sufficient visibly clear the LAL without damaging the enzymes. In some embodiments, the term “clarified LAL” refers to LAL that has been centrifuged at 1800 to 8000 g, 2200 g to 7600 g, 2600 g to 7200 g, 3000 g to 7200 g, 3400 g to 7200 g, 3800 g to 7200 g, 4200 g to 7200 g, 4600 g to 7200 g, 5000 g to 7200 g, 5400 g to 7200 g, 5800 g to 7200 g, or 6100 g to 7200 g for a period of time sufficient visibly clear the LAL without damaging the enzymes.

In some embodiments, the term “clarified limulus amebocyte lysate” (or “clarified LAL”) that is substantially free of coagulogen refers to LAL substantially free of coagulogen, discussed above, that has been further treated to remove components that create a cloudy appearance of the LAL by the centrifuging LAL substantially free of coagulogen at greater than 20,000×g, greater than 22,000×g, greater than 24,000×g, greater than 25,000×g, greater than 26,000×g, greater than 28,000×g, greater than 30,000×g, greater than 35,000×g, greater than 40,000×g, greater than 45,000×g or greater than 50,000×g. In some embodiments, the LAL substantially free of coagulogen is centrifuged a at greater than 20,000-50,000×g, 20,000-40,000×g, 25,000-50,000×g, 25,000-40,000×g, or 30,000-40,000×g. In some embodiments, the LAL substantially free of coagulogen is centrifuged for greater than 20 minutes, greater than 30 minutes, greater than 40 minutes or greater than 60 minutes. In some embodiments, the LAL substantially free of coagulogen is centrifuged for 20-120 minutes, 20-90 minutes, 20-60 minutes, 20-40 minutes or about 30 minutes.

In some embodiments, the term “clarified LAL” refers to LAL that has been centrifuged for greater than 3 minutes, greater than 4 minutes, greater than 5 minutes, greater than 6 minutes, greater than 7 minutes, greater than 8 minutes, greater than 9 minutes, or greater than 10 minutes. In some embodiments, the term “clarified LAL” refers to LAL that has been centrifuged for 3 minute to 30 minutes, 4 minutes to 25 minutes, 4 minutes to 20 minutes, 5 minutes to 15 minutes or 5 minutes to 10 minutes. One of skill in the art can appreciate that a lower speed of centrifugation may require a longer centrifugation time, and will adjust the time and/or speed accordingly to reduce the visual cloudiness of the LAL. In some embodiments, the term “clarified LAL” refers to LAL substantially free of coagulogen centrifuged at about 5000 g to about 7000 g for about 3 minutes to about 10 minutes, or about 6120 g for 5 minutes. In embodiments, clarified LAL substantially free of coagulogen is made by centrifuging a solution derived from lysed amebocytes from Limulus polyphemus at 2,000 rpm (980 g) for 8 minutes at 4° C. The clarified LAL is found in the supernatant after centrifugation. In some embodiments the resulting supernatant is then combined with a buffer; the resulting combination of supernatant and buffer is then subjected to tangential flow filtration using a 30 kDa membrane filter to produce a retentate; and the retentate is centrifuged at 5,000 rpm (6120 g) for 5 minutes at 4° C. to produce a supernatant, wherein the supernatant is clarified LAL that is substantially free of coagulogen. In embodiments, the solution derived from lysed amebocytes from Limulus polyphemus is a pool of multiple Limulus polyphemus lysed amebocytes.

In some embodiments, the hemocyte lysate dried on the optical sample well is obtained by obtaining a solution derived from lysed amebocytes from Limulus polyphemus. In some embodiments, the solution is then combined with a buffer; the resulting combination of solution and buffer is then subjected to continuous tangential flow filtration (TFF) using a 20 kDa to 50 kDa membrane filter to produce a retentate; and the retentate is centrifuged at greater than 20,000×g for greater than 25 minutes at 4° C. to produce a supernatant, wherein the supernatant is clarified LAL that is substantially free of coagulogen. In embodiments, the lysate is derived from lysed amebocytes from Limulus polyphemus or a pool of multiple Limulus polyphemus lysed amebocytes. In some embodiments, the continuous TFF comprises at least four diafiltration volumes (DV). In some embodiments, the continuous TFF comprises at least five diafiltration volumes. In some embodiments, the continuous TFF comprises at least six diafiltration volumes. In some embodiments, the continuous TFF comprises at least 7, at least 8, at least 9, at least 10, at least 11, at least 12, at least 13, at least 14, or at least 15 diafiltration volumes.

In some embodiments, clarified LAL substantially free of coagulogen according to the present disclosure is produced by a method utilizing any combination of the technical features described herein. Thus, one of skill in the art can use any of the listed filters, filter sizes, filter flow rates, buffers, centrifugation speeds, centrifugation temperatures, centrifugation times, etc., sufficient to make the LAL substantially free of coagulogen. For example, in some embodiments, the LAL (i) is centrifuged at greater than 20,000×g, greater than 22,000×g, greater than 24,000×g, greater than 25,000×g, greater than 26,000×g, greater than 28,000×g, greater than 30,000×g, greater than 35,000×g, greater than 40,000×g, greater than 45,000×g or greater than 50,000×g, (ii) is centrifuged at a temperature of 2° C. to 10° C., 2° C. to 8° C., or 4° C., (iii) is centrifuged for 20-120 minutes, 20-90 minutes, 20-60 minutes, 20-40 minutes or about 30 minutes, (iv) undergoes TFF at a flow rate of greater than 500 mL/min, e.g., 500 mL/min to 2000 mL/min, 800 mL/min to 1500 mL/min, or 1000 mL/min to 1200 mL/min, (v) undergoes TFF using a 50 kDa filter, a 45 kDa filter, a 40 kDa filter, a 35 kDa filter, a 30 kDa filter, a 25 kDa filter, or a 20 kDa filter, (vii) undergoes TFF using at least 4 DV, at least 5 DV, at least 6 DV, at least 7 DV, or at least 8 DV, etc.

The present application further provides for a hemocyte lysate, e.g., LAL or clarified LAL, wherein the composition is made by a method comprising: centrifuging a solution derived from lysed amebocytes from Limulus polyphemus at 2,000 rpm for 8 minutes at 4° C. to produce a supernatant; combining the supernatant from (a) with a buffer; subjecting the combination from (b) to tangential flow filtration using a 30 kDa membrane filter to produce a retentate; and centrifuging the retentate from (c) at 5000 rpm (e.g., 6120 g) for 5 minutes at 4° C. to produce a supernatant, wherein the supernatant is clarified LAL that is substantially free of coagulogen.

In some embodiments, the hemocyte lysate dried on the optical sample well disclosure is made by a method comprising centrifuging a solution derived from lysed amebocytes, e.g., from Limulus polyphemus, at 1000 to 3000 rpm for 2 to 15 minutes at 2 to 10° C. to produce a first supernatant (“the first centrifuging”); combining the supernatant with a buffer; filtering the combination using a 20 kDa to 50 kDa filter to produce a retentate; centrifuging the retentate at 3000 to 7000 rpm for 2 to 10 minutes at 2 to 10° C. to produce a second supernatant (“the second centrifuging”), wherein the second supernatant comprises clarified hemocyte lystate, e.g., LAL, that is substantially free of coagulogen. In embodiments, the filtering is subjecting the hemocyte lysate, e.g., LAL, to TFF. In some embodiments, then hemocyte lysate, e.g., LAL, is placed in a buffer prior to TFF. In some embodiments, the buffer is a Tris buffer or MES buffer. In some embodiments, the buffer has a pH of about 6.0 to about 9.0, or about 7.0 to about 8.0. In embodiments, the first centrifuging comprises centrifuging at 2000 rpm. In embodiments, the first centrifuging comprises centrifuging for 8 minutes. In embodiments, the first centrifuging comprises centrifuging at 4° C. In embodiments, the second centrifuging comprises centrifuging at 5000 rpm. In embodiments, the second centrifuging comprises centrifuging for 5 minutes. In embodiments, the second centrifuging comprises centrifuging at 4° C.

Various membranes can be used in the TFF. Filters of varying pore sizes can be used in TFF, depending on the size of the desired protein to be reduced in the resulting retentate. In the present disclosure, Factor C, Factor B, Factor G and proclotting enzyme are known to be involved in the clotting cascade system of hemocyte lysate, e.g., LAL, resulting in the conversion of coagulogen into an insoluble coagulin gel. For purposes of the disclosure provided herein, any TFF procedure (and accompanying filter pore size, pore type and buffer system) can be used which results in coagulogen being reduced, and Factor C, Factor B, Factor G and proclotting enzyme being retained. Thus, in some embodiments, the TFF procedure uses a 50 kDa filter, a 45 kDa filter, a 40 kDa filter, a 35 kDa filter, a 30 kDa filter, a 25 kDa filter, or a 20 kDa filter. In some embodiments, a 40 kDa to a 25 kDa filter is used. In some embodiments, the membrane is a 10 to 80 kDa filter, or a 20 to 50 kDa filter. In some embodiments, the filter is a 30 kDa filter.

The membranes use in the method disclosed herein can include, but are not limited to modified Polyethersulfone (mPES), Polysulfone (PS) and Polyethersulphone (PES). In some embodiments, making hemocyte lysate, e.g., LAL, substantially free of coagulogen is performed using TFF using a modified polyethersulfone (mPES) membrane filter. The rate of flow of the hemocyte lysate, e.g., LAL, across the membranes can be adjusted to optimize removal of the coagulogen from the hemocyte lysate, e.g., LAL. In some embodiments, the TFF is performed at a flow rate of 200 mL/min to 800 mL/min, 300 mL/min to 600 mL/min, or 350 mL/min to 500 mL/min. In some embodiments, the TFF is performed at a flow rate of greater than 500 mL/min, e.g., 500 mL/min to 2000 mL/min, 800 mL/min to 1500 mL/min, or 1000 mL/min to 1200 mL/min. In some embodiments, the TFF is performed at 1000 mL/min, 1100 mL/min, 1200 mL/min, 1300 mL/min or 1400 mL/min. In some embodiments, the TFF is performed at 1100 mL/min.

Divalent metal salts, which are known to promote activation of the pro-clotting enzyme of hemocyte lysate, as well as buffers to avoid extremes of pH that could inactivate the clotting enzyme can also be included in the dried composition. Various buffers and salts that are understood in the art to be compatible with the amoebocyte lysate system may be used. Typical formulation additives may include, without limitation, NaCl (about 100-300 mM NaCl), about 10-100 mM divalent cations (e.g., Mg²⁺ or Ca²⁺), biocompatible buffers, e.g., Tris (tris(hydroxy)aminomethane), to give a final pH of about 6.0 to about 8.0.

In addition, to facilitate drying of the lysate, various stabilizers such as sugars, e.g., mannitol, sucrose, trehalose, dextran, etc. can be added to aid in lyophilization or vitrification.

Synthetic chromogenic substrates have been used to measure the level of endotoxin-activated pro-clotting enzyme in LAL prepared from the hemolymph of both Tachypleus tridentatus and Limulus polyphemus horseshoe crabs (Iwanaga et al. (1978) Hemostasis 7: 183-188). During an LAL assay that uses a chromogenic substrate, the pro-clotting enzyme (a serine protease) in the LAL is activated by endotoxin and cleaves the substrate's peptide chain on the carboxyl side of arginine so as to release the chromogenic group from the substrate, thereby releasing a marker compound that can be easily detected by, for example, spectrophotometry. One advantage of using a synthetic chromogenic substrate in an LAL assay in place of a conventional LAL gelation test is that the amount of activated clotting enzyme can be quantified and correlated to endotoxin levels in the sample.

Any chromogenic substrate that is cleaved by the clotting enzyme of a hemocyte lysate may be used in the cartridges, methods and compositions described herein. U.S. Pat. No. 5,310,657, for example, describes an exemplary chromogenic substrate having the formula R₁-A₁-A₂-A₃-A₄-B—R₂, where R₁ represents hydrogen, a blocking aromatic hydrocarbon or an acyl group; A₁ represents an L or D-amino acid selected from Ile, Val or Leu; A₂ represents Glu or Asp; A₃ represents Ala or Cys; A₄ represents Arg; B represents a linkage selected from an ester and an amide; and R₂ represents a chromogenic or fluorogenic group which is covalently attached to the C-carboxyl terminal of Arginine through the B linkage, the fluorogenic or chromogenic moiety being capable of being cleaved from the remainder of the chromogenic substrate to produce a chromogen or a fluorogen. An exemplary chromogenic substrate has the consensus sequence acetate-Ile-Glu-Ala-Arg-pNA, where pNA represents a para-nitroaniline group. U.S. Pat. No. 4,188,264 describes a peptide substrate with a structure consisting of L-amino acids in the sequence R₁-Gly-Arg-R₂ where R₁ represents an N-blocked amino acid and R₂ is a group that can be released by enzymatic hydrolysis to yield a colored compound, HR₂. U.S. Pat. No. 4,510,241 discloses a chromogenic peptide substrate, which differs from the previous substrate in that the Gly moiety is replaced in the sequence by Ala or Cys. Alternatively, the chromogenic substrate may contain a fluorophore, for example, 7-amino-4-methyl coumarin, 7-amino-4-trifluoromethyl coumarin, and 4-methoxy-2-naphthalyamine.

Various concentrations of chromogenic substrates can be used. In some embodiments, the chromogenic substrate has a concentration of 0.1 g/l to 0.5 g/l, 0.1 g/l to 0.4 g/l, 0.2 g/l to 0.4 g/l, 0.2 g/l to 0.3 g/l or 0.2 g/l to 0.25 g/l.

Inhibition or enhancement of the assay occurs when substances in the test sample interfere with the hemocyte lysate reaction. Inhibition results in a longer reaction time, indicating lower levels of microbial contamination than may actually be present in the test sample. Enhancement results in shorter reaction time, indicating higher levels of microbial contamination than may actually be present in the test sample.

Exemplary amounts of hemocyte lysate, chromogenic substrate and/or an agent representative of a microbial contaminant that can dried on the optical sample well are described herein or otherwise readily determined by those of ordinary skill in the art. In some embodiments, the dried compositions comprise amounts of the components such that a ratio of about 30% to 50% hemocyte lysate, and 10% to 30% chromogenic substrate (v/v), are provided. In some embodiments, the dried composition comprise amounts of the components such that a ratio of about 35% to about 45% hemocyte lysate and 15% to 25% chromogenic substrate, or about 40% hemocyte lysate, and 20% chromogenic substrate (wt/wt), are provided. In other embodiments, the dried compositions comprise amounts of the components such that a ratio of about 30% to 50% LAL substantially free of coagulogen, and 10% to 30% chromogenic substrate (v/v), are provided. In some embodiments, the dried composition comprises amounts of the components such that a ratio of about 35% to about 45% LAL substantially free of coagulogen and 15% to 25% chromogenic substrate, or about 40% LAL substantially free of coagulogen, and 20% chromogenic substrate (wt/wt), are provided. In other embodiments, the dried compositions comprise amounts of the components such that a ratio of about 30% to 50% clarified LAL, and 10% to 30% chromogenic substrate (v/v), are provided. In some embodiments, the dried composition comprise amounts of the components such that a ratio of about 35% to about 45% clarified LAL and 15% to 25% chromogenic substrate, or about 40% clarified LAL, and 20% chromogenic substrate (wt/wt), are provided.

In some embodiments, the dried compositions comprises about 1 μg to about 50 μg hemocyte lysate, and about 0.1 μg to 5 μg chromogenic substrate, about 1 μg to about 30 μg hemocyte lysate, and about 0.5 μg to 4.0 μg chromogenic substrate, or about 2 μg to about 20 μg hemocyte lysate, and about 1.0 μg to about 3.0 μg chromogenic substrate, or about 4 μg to about 25 μg hemocyte lysate, and about 1.0 μg to about 2 μg chromogenic substrate. In some embodiments, the dried compositions comprises about 1 μg to about 50 μg LAL substantially free of coagulogen and about 0.1 μg to about 5 μg chromogenic substrate, or about 1 μg to about 30 μg LAL substantially free of coagulogen and about 0.5 μg to about 5 μg chromogenic substrate, or about 2 μg to about 20 μg LAL substantially free of coagulogen and about 1.0 μg to about 3.0 μg chromogenic substrate, or about 1 μg to about 30 μg LAL substantially free of coagulogen, and about 1.0 μg to about 2.0 μg chromogenic substrate, wherein the chomogenic substrate is Ac-Ile-Glu-Ala-Arg-pNA. In some embodiments, the dried compositions comprises about 4 μg to about 25 μg LAL substantially free of coagulogen, and about 1 μg to about 1.5 μg chromogenic substrate, wherein the chomogenic substrate is Ac-Ile-Glu-Ala-Arg-pNA. In some embodiments, the dried compositions comprises about 1 μg to about 50 μg clarified LAL and about 0.1 μg to about 5 μg chromogenic substrate, or about 1 μg to about 30 μg clarified LAL and about 0.5 μg to about 5 μg chromogenic substrate, or about 2 μg to about 20 μg clarified LAL and about 1.0 μg to about 3.0 μg chromogenic substrate, or about 1 μg to about 30 μg clarified LAL, and about 1.0 μg to about 2.0 μg chromogenic substrate, wherein the chomogenic substrate is Ac-Ile-Glu-Ala-Arg-pNA. In some embodiments, the dried compositions comprises about 4 μg to about 25 μg clarified LAL, and about 1 μg to about 1.5 μg chromogenic substrate, wherein the chomogenic substrate is Ac-Ile-Glu-Ala-Arg-pNA.

In some embodiments, the microbial contaminant control is about 0.1 EU/ml to 1 EU/ml. In some embodiments, the microbial contaminant is a bacterial endotoxin in a concentration of about 0.1 EU/ml to 1 EU/ml, wherein 1 μl to 10 μl is used.

In suitable embodiments, a formulation containing about 10% to about 60% hemocyte lysate or about 20% to about 50% hemocyte lysate or about 30% to about 40% hemocyte lysate, or about 10% to about 60% LAL substantially free of coagulogen or about 20% to about 50% LAL substantially free of coagulogen or about 30% to about 40% LAL substantially free of coagulogen is deposited onto optical sample well 104, prior to lyophilizing to yield the dried composition. In some embodiments, a formulation containing about 10% to about 60% clarified LAL or about 20% to about 50% clarified LAL or about 30% to about 40% clarified LAL is deposited onto optical sample well 104, prior to lyophilizing to yield the dried composition. The volume of the hemocyte lysate, LAL substantially free of coagulogen formulation, or clarified LAL formulation that is deposited prior to lyophilization will generally be on the order of about 1 μL to about 10 μL. In further embodiments, the formulation can contain about 20% to about 30%, or about 25% to about 30% hemocyte lysate or LAL substantially free of coagulogen, with a deposited volume of about 3 μL to about 10 μL, or about 3 μL to about 8 μL, or about 3.5 μL to about 7 μL. In further embodiments, the formulation can contain about 20% to about 30%, or about 25% to about 30% clarified LAL, with a deposited volume of about 3 μL to about 10 μL, or about 3 μL to about 8 μL, or about 3.5 μL to about 7 μL. In embodiments, the volume of clarified LAL that is deposited in each well is suitably within about 10% (by volume) from well-to-well, suitably less than a variation of about 5% from well-to-well.

Cartridge 100 can also include pH indicator 105, such as pH paper or other suitable compound or composition which can be used to directly determine the pH of the sample. As shown in FIG. 1B (pH indicator 105 is not shown in FIG. 1A for ease of viewing), in embodiments, pH indicator 105 is directly associated with optical sample well 104, but can also be associated with conduit 108, depending on the orientation of cartridge 100. In either embodiment, pH indicator 105 can be readily used to measure sample pH. Cartridge 100 can also include a bar code 120, useful for identifying the cartridge for ease in storage and automated data collection, etc.

In additional embodiments, for example as shown in FIGS. 1A-1B, housing 102 includes four optical sample wells, though other or additional numbers of optical sample wells, e.g., 2, 3, 4, 5, 6, 7, 8, 9, 10, etc., can be utilized. Suitably, each optical sample well includes the dried composition including the hemocyte lysate. Each optical sample well suitably further includes a chromogenic substrate, as described herein, dried on the optical sample wells, as an element of the dried composition.

In embodiments, two of the four optical sample wells further include an agent representative of the microbial contaminant dried on the optical sample wells, which can act as a control to verify that the methods described herein are functioning correctly, as are the various substrates, lysates, etc. In other embodiments, housing 102 can include 2 optical sample wells, where one sample well acts as a control and the other a test well, or can include 4, 6, 8, 10, 12, 14, etc., where some portion of the optical sample wells (e.g., 2, 4, 6, 8, etc.) functioning as controls, with the other optical sample wells, function as test wells.

To verify the lack of inhibition or enhancement, control optical sample wells are suitably “spiked” with a known amount of an agent representative of the microbial contaminant to be measured. Suitably, the microbial contaminant spike results in a final microbial contaminant concentration in the sample near to the mid-point, on a log basis, between the microbial contaminant concentration of the highest and lowest standards in a standard curve. For example, in an assay with a standard curve spanning from 50 Endotoxin Units (EU)/mL to 0.005 EU/mL, samples can be spiked to contain a final microbial contaminant concentration of about 0.5 EU/mL. In an assay with a standard curve spanning from 1 EU/mL to 0.01 EU/mL, the microbial contaminant spike can result in a final microbial contaminant concentration of about 0.1 EU/mL.

Suitably, the “spike,” or known amount of an agent representative of the microbial contaminant to be measured, is dried onto the optical sample well in a section or area of the well that is distinct from the dried composition. For example, as shown in FIG. 1C, dried composition 132 can be on bottom 136 of optical sample well 104, with agent representative of the microbial contaminant 134 on top 138 of the optical sample well.

The spiked sample(s) is assayed in parallel with the unspiked, or test sample. The resulting microbial contaminant concentration in the unspiked sample and the microbial contaminant recovered in the spiked sample then are calculated and compared. The microbial contaminant recovered suitably equals the known concentration of the spike within about 25%. If the sample (or dilution) is found to inhibit or enhance the reaction, the sample may require further dilution until the inhibition or enhancement is overcome. Initially, it may be desirable to screen for inhibition or enhancement by testing 10-fold dilutions of the sample.

In additional embodiments dried composition 132 can include various other reagents/reactants, such that the cartridges described herein can be used for additional reactions. In such embodiments, the ability to introduce the sample into optical sample wells 104, in which a pre-determined amount of desired reactants (e.g., buffers, enzymes, stabilizers, etc.) are included as lyophilized or vitrified dried compositions, provides a platform for various additional testing opportunities beyond endotoxin detection.

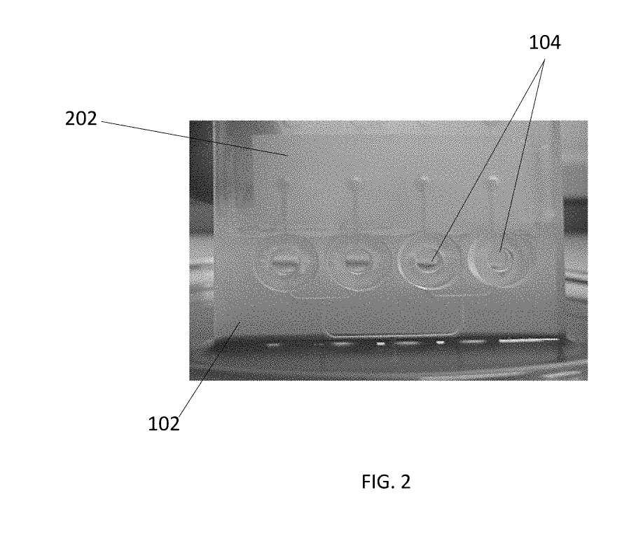

In embodiments, as shown in FIG. 2, housing 102 includes liquid impermeable membrane 202, which aids with filling and maintaining the sample volume in optical sample wells 104. As used herein “liquid impermeable membrane” refers to a substrate that allows air to pass through the substrate, but is largely impermeable to liquids, and suitably does not allow any liquids to pass through the membrane. Examples of such membranes include various rubbers and polymers, including for example, poly(propylene) membranes, poly(tetrafluoroethylene) (PTFE) membranes, other fluoropolymers, etc. Suitably, liquid impermeable membrane 202 is fluidly connected to optical sample well 104, such that the liquid sample that fills the optical sample well, comes in contact with the liquid impermeable membrane, and stops flowing as it impacts liquid impermeable membrane, and thus maintains the volume(s) of sample in each of the optical sample wells such that they are the same (or within about 1-10% of the same volume), so that each sample can be compared with the other.

Housing 102 is suitably prepared from two individual sections, top section 302 and bottom section 304, that are mechanically connected to each other to form cartridge 100. Utilizing two sections connect together allows for easier preparation of the various parts of cartridge 100, as well as assembly. For example, channels, microchannels, or tubes that make up conduit 108, fluid inlet port 106, as well as optical sample wells 104, can be pre-formed or pre-placed into the top and/or bottom sections, and then connected together to form the finished cartridge 100. Methods for mechanically connecting top 302 and bottom 304 sections include various adhesives or glues, laser welding, ultrasonic welding mechanical screws or self-fitting connectors, as well as bands or clips. In additional embodiments, the mechanical connection can be carried out by thermal fusion or thermal bonding between the two sections. In embodiments, top 302 and bottom 304 sections of housing 102 can be injection molded and then thermally bonded or fusion bonded to each other, thereby suitably enclosing conduit 108, optical sample wells 104 and fluid inlet port 106. In further embodiments, one of top 302 and bottom 304 sections of housing 102 can be prepared using an opaque plastic, which reduces unwanted light passing through the housing during measurements, reducing cross-talk.

In additional embodiments, provided herein are cartridges for determining the presence and/or amount of a microbial contaminant in a sample. Cartridge 100 suitably includes housing 102 including first and second optical sample wells 104, fluid inlet port 106 and conduit 108 fluidly connecting the fluid inlet port and the first and the second optical sample wells.

The cartridge also suitably includes a two-position syringe attached to housing 102 and fluidly connected to fluid inlet port 106 and conduit 108. In embodiments, as described herein, a first position creates a vacuum to introduce the sample into fluid inlet port 106 and conduit 108, and a second position provides transport from conduit 108 to optical sample wells 104. Also included in the cartridge is dried composition 132 including a hemocyte lysate (suitably limulus amoebocyte lysate) and a chromogenic substrate dried on each of the optical sample wells, as well as an agent representative of the microbial contaminant dried on the second optical sample well.

In embodiments, housing 102 includes four optical sample wells, each comprising the dried composition dried on the optical sample wells, with two of the four optical sample wells including the agent representative of the microbial contaminant dried on the optical sample wells. As described herein, these two optical sample wells include the “spike” of microbial contaminant (e.g., a bacterial endotoxin) that is used as a control for the determination of the presence and/or amount of the microbial contaminant.

As described herein, in embodiments, housing 102 includes liquid impermeable membrane 202 fluidly connected to each of the optical sample wells. Liquid impermeable membrane 202 provides a mechanism for stopping the flow of the sample as it fills the optical sample wells, thereby maintaining the volumes of sample in each of the optical sample wells such that they are the same (or within about 1-10% of the same volume), so that each sample can be compared with the other. As described throughout, suitably housing 202 includes top 302 and bottom 304 sections, that are mechanically connected to each other so as to form the cartridge.

Also provided herein are methods for detecting the presence of a microbial contaminant in a sample. In embodiments, the methods suitably include introducing the sample into the fluid inlet port of the cartridges described herein. For example, as shown in FIG. 1D, sample 170 can be held in a container, or can be pulled directly from a reaction process or batch, or a pharmaceutical solution. Suitably, sample 170 is introduced via sample introduction direction 114 by creating a vacuum via pump mechanism 110, suitably a two-position syringe, to introduce the sample into the fluid inlet port. The sample is also transferred to conduit 108 in this initial process, also suitably via the vacuum that is produced by pump mechanism, for example by placing a two-position syringe in first position 124. As shown in FIG. 1A, sample 170 is transferred to conduit 108, such that it fills substantially all of conduit 108 to an initial position, but does not begin to fill optical sample well 104.

Then, pump mechanism, suitably a two-position syringe, placed in second position 126, transfers sample 170 from conduit 108 to optical sample well 104, as shown in FIG. 1B. As described herein, the transporting of the sample is stopped by liquid impermeable membrane 202, which is fluidly connected to the optical sample well. As described herein, stopping the transporting allows each of the optical sample wells to fill substantially equally in volume (such that the volumes vary within about 10% of each other, suitably about 5%, or about 1%) to allow for comparison between the optical sample wells.

The methods further include measuring an optical property of the sample in the optical sample well. In the methods of detecting the presence of the microbial contaminant, a change in the optical property is indicative of the presence of the microbial contaminant in the sample.

As described herein, the optical property is suitably the absorbance of the sample at a preselected wavelength of light, and the change in the optical property is the change in absorbance. The optical property measured can be a change (e.g., an increase or decrease) in the absorbance at a particular wavelength, transmittance at a particular wavelength, fluorescence at a particular wavelength, or optical density. For example, the optical property may be a change in absorbance or transmittance at a wavelength in the range from about 200 nm to about 700 nm, and more suitably in the range from about 350 nm to about 450 nm, or about 400 nm to about 410 nm, or about 405 nm.

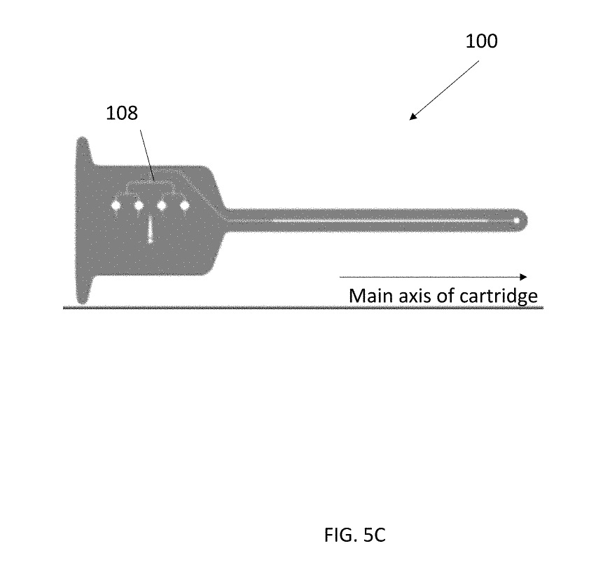

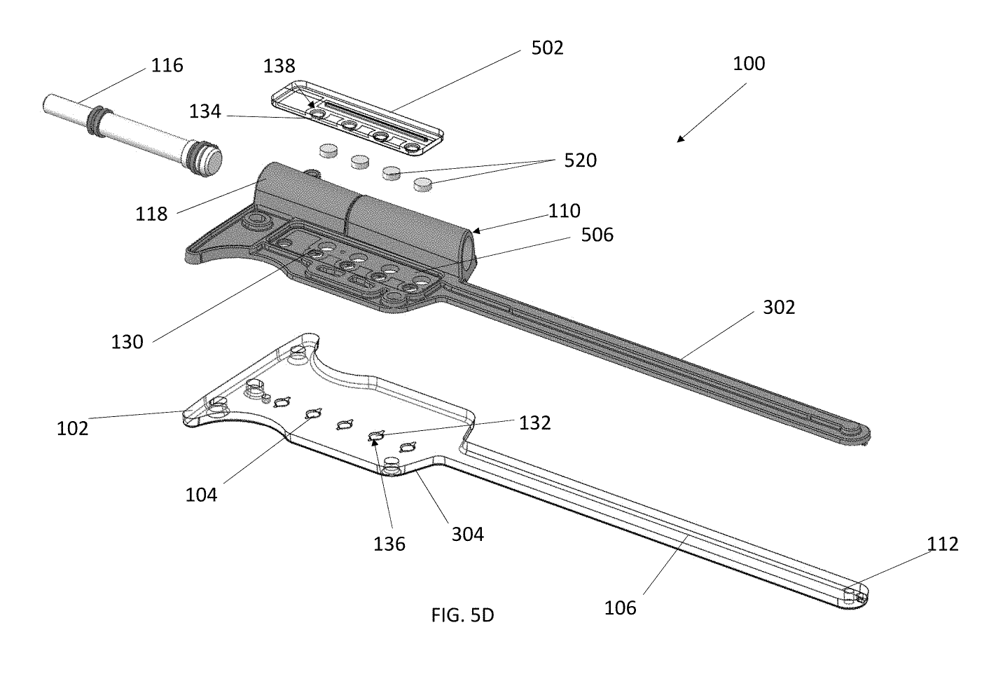

FIG. 5A shows an additional cartridge 100 configuration in accordance with embodiments described herein. As described herein, housing 102 is suitably prepared from two individual sections, top section 302 and bottom section 304, that are connected to each other to form cartridge 100. Bottom section 304 of cartridge 100 includes bottom 136 of optical sample well 104, with dried composition 132 dried therein. Top section 302 includes openings that form well walls 130, while an upper plate 502 (suitably a clear polymeric element, prepared from polystyrene or other polymeric material) includes top 138 of sample wells 104 with agent representative of the microbial contaminant 134 dried on top 138 (see configuration in FIG. 1C).

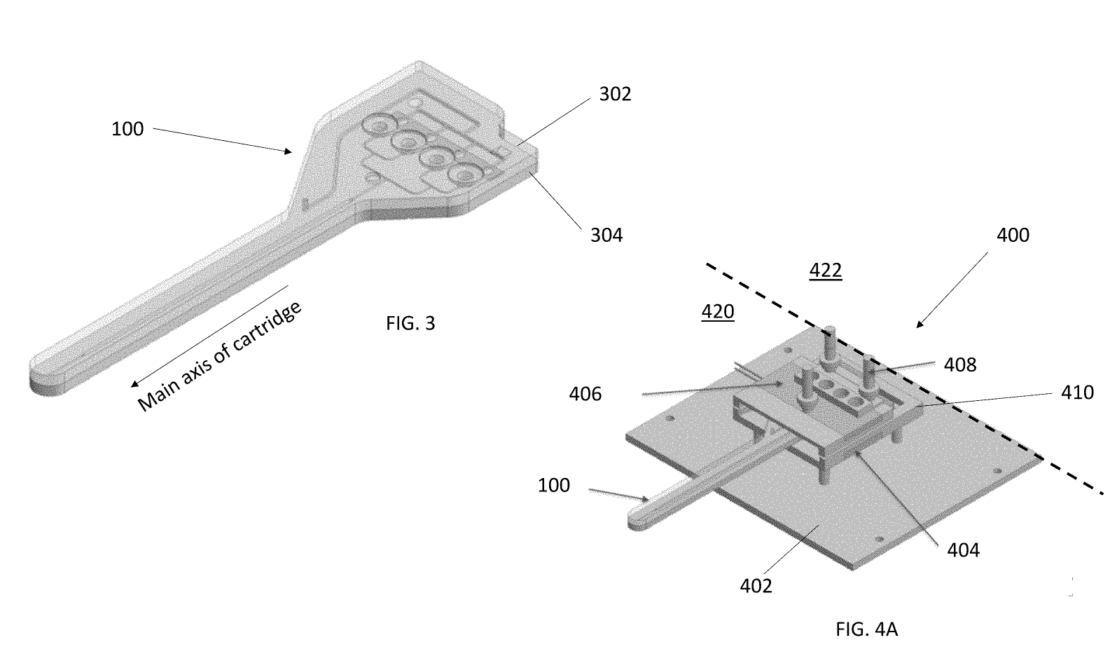

Top section 302, suitably adjacent sample wells 104, includes an indented section 506, within which liquid impermeable membrane 202 can sit, fluidly connected to sample wells 104, so as to stop the flow of liquid into sample wells 104, as the wells fill from conduit 108, as described herein. (See FIG. 5C for location of conduit 108 in this embodiment of cartridge 100, as well as the orientation of sample wells 104, which suitably are aligned with the main axis of cartridge 100. See FIG. 3 for an alternative orientation of sample wells 104, which are perpendicular to the main axis of the cartridge.) Also included is a silicone backing 504 which provides additional sealing and integration as upper plate 502 is added to the cartridge. FIG. 5B shows an assembled view of cartridge 100, where the position of upper plate 502, sealing against bottom section 304 of cartridge 100, and sitting within top section 302.

FIG. 5 A shows the location of fluid inlet port 106, as well as pump mechanism 110, which includes barrel 118 integrated directly onto top section 302 of cartridge 100, into which plunger 116 can be inserted (see FIG. 5B showing plunger 116 inserted into barrel 118.

FIG. 5D shows a still further, additional cartridge 100 configuration in accordance with embodiments described herein. As described herein, housing 102 is suitably prepared from two individual sections, top section 302 and bottom section 304, that are connected to each other to form cartridge 100. Bottom section 304 of cartridge 100 includes bottom 136 of optical sample well 104, with dried composition 132 dried therein. Top section 302 includes openings that form well walls 130, while an upper plate 502 (suitably a clear polymeric element, prepared from polystyrene or other polymeric material) includes top 138 of sample wells 104 with agent representative of the microbial contaminant 134 dried on top 138 (see configuration in FIG. 1C).

Top section 302, suitably adjacent sample wells 104, can also include an indented section 506. In embodiments, liquid impermeable membrane, in the form of individual impermeable membranes 520, are utilized and fluidly connected to sample wells 104, so as to stop the flow of liquid into sample wells 104, as the wells fill from conduit 108, as described herein. (See FIG. 5C for location of conduit 108 in such an embodiment of cartridge 100, as well as the orientation of sample wells 104, which suitably are aligned with the main axis of cartridge 100. See FIG. 3 for an alternative orientation of sample wells 104, which are perpendicular to the main axis of the cartridge.) Use of individual impermeable membranes 520, for example in the form of 2-10, suitably 4, circular or similarly-shaped disks, as the liquid impermeable membrane which can be individually pressed into well walls 130, provide for a snug fit and good control of liquid flow. Individual impermeable membranes are suitably poly(tetrafluoroethylene) (PTFE) membranes with a size on the order of about a few millimeters in diameter and a few millimeters in thickness, suitably about 1 mm in thickness by about 2 mm in diameter.

As described herein, an exemplary mechanism for sealing top section 302 and bottom section 304 of cartridge 100 includes laser welding and ultrasonic welding. As shown in FIG. 5A, suitably top section 302 is prepared from a polymeric material, such as polystyrene, which contains a small amount (e.g., 0.5-10%, suitably 2-5% by weight) of carbon black. Inclusion of carbon black in top section 302 allows for top section 302 to be laser welded to bottom section 304, which is suitably a clear, polymeric material, such as polystyrene. Methods for carrying out the laser welding are known in the art, for example as disclosed in Klien, “Laser Welding of Plastics,” Wiley-VCH, Germany (2012), the disclosure of which is incorporated by reference herein in its entirety. Use of laser welding reduces concerns related to heat-caused degradation of liquid impermeable membrane, either as the single membrane 202 or individual impermeable membranes 520.

Methods of ultrasonic welding are well known in the art, for example as disclosed in Shoh, “Welding of thermoplastics by ultrasound,” Ultrasonics 14: 209-217 (1976), the disclosure of which is incorporated by reference herein in its entirety. Use of ultrasonic welding reduces concerns related to heat-caused degradation of liquid impermeable membrane, either as membrane 202 or individual impermeable membranes 520.

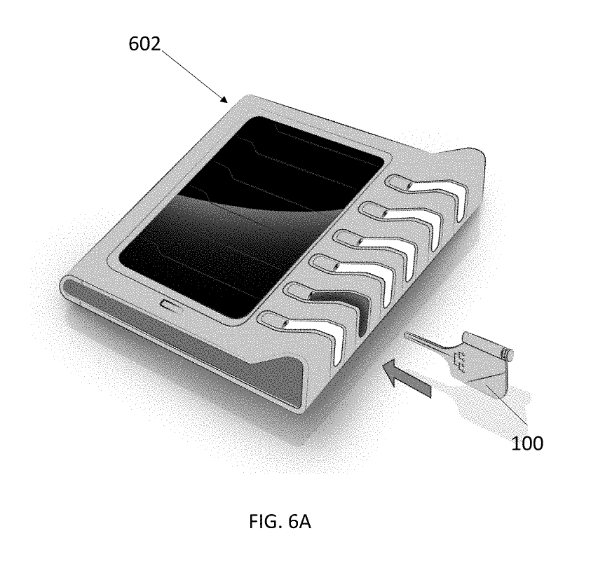

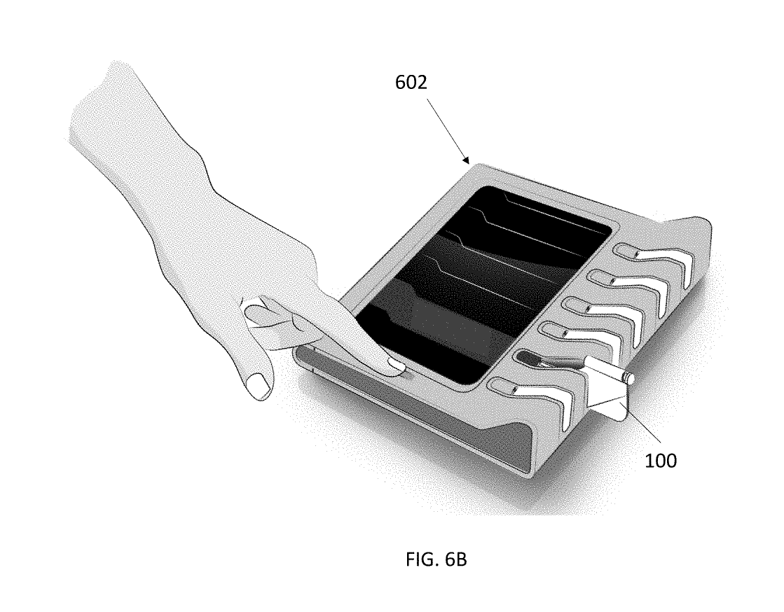

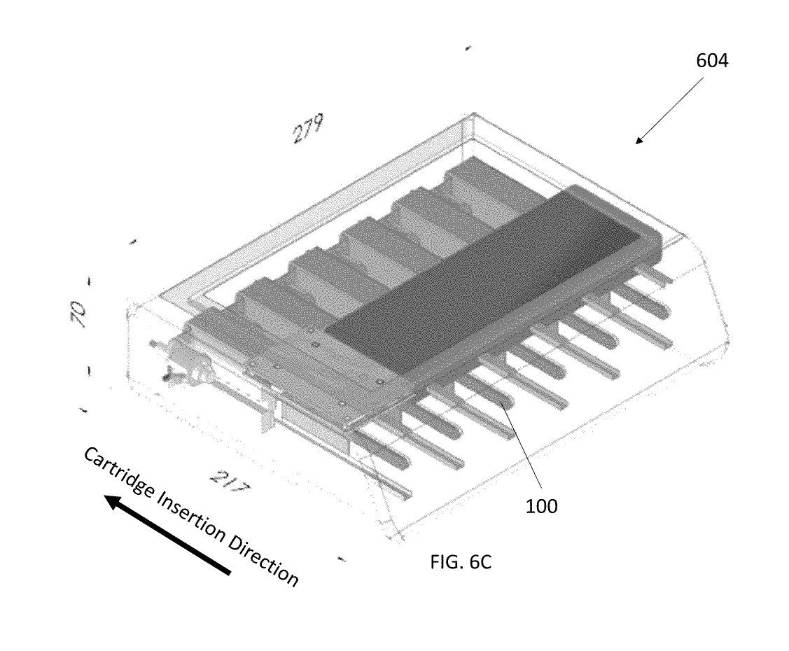

In exemplary embodiments, the cartridges described herein, containing the sample in optical sample wells 104, can be inserted in a measurement device or reader device 602, such as show in FIG. 6A. It should be noted that reader device 602 in FIG. 6A is provided only for illustrative purposes, and does not limit the scope of the invention, including how the cartridges described herein are read or analyzed.

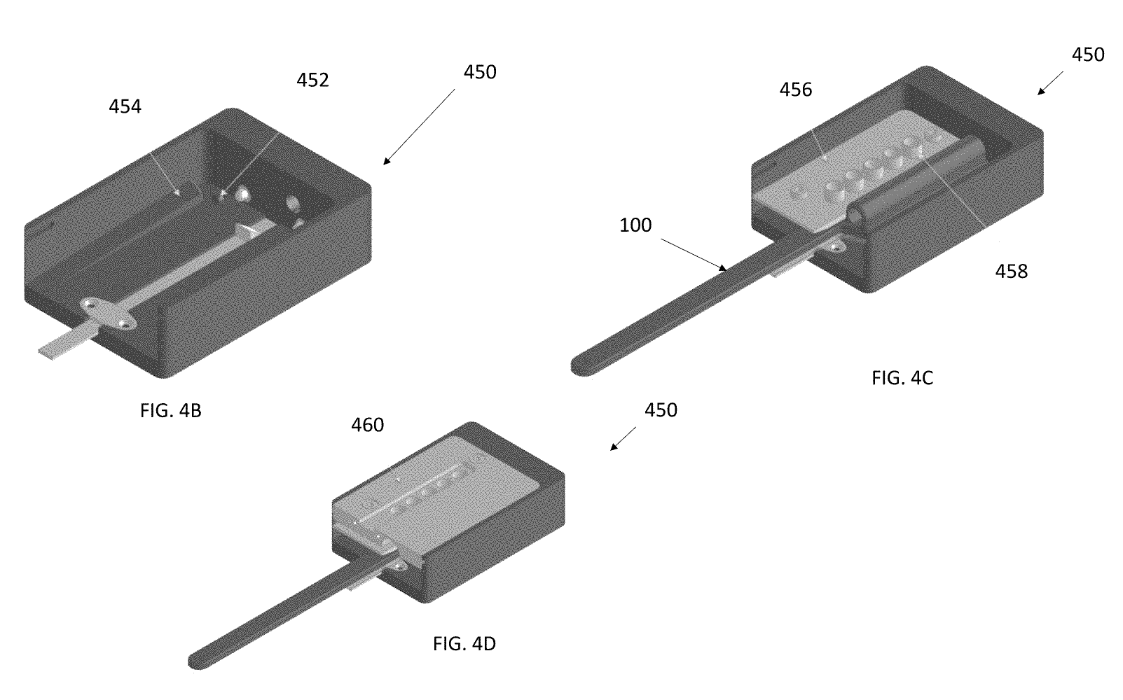

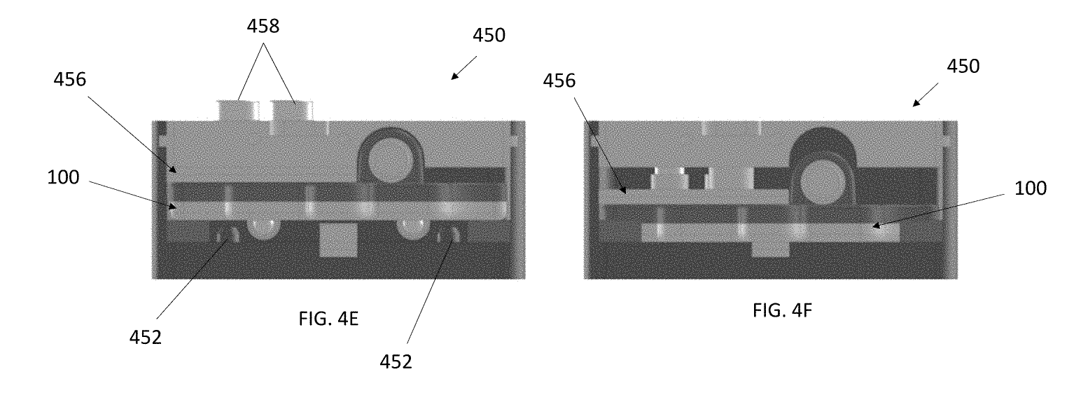

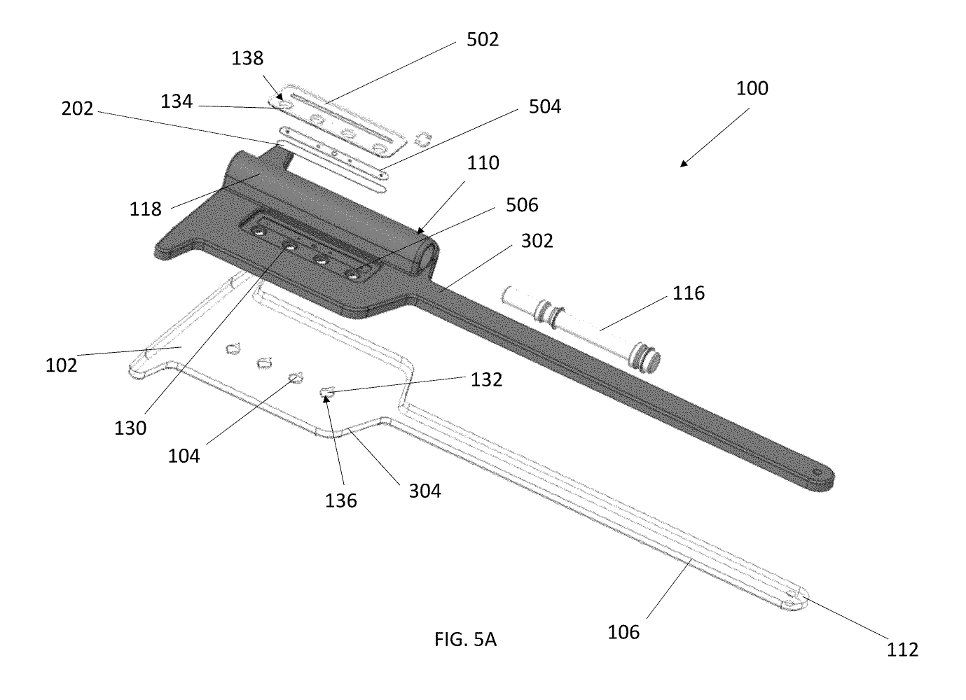

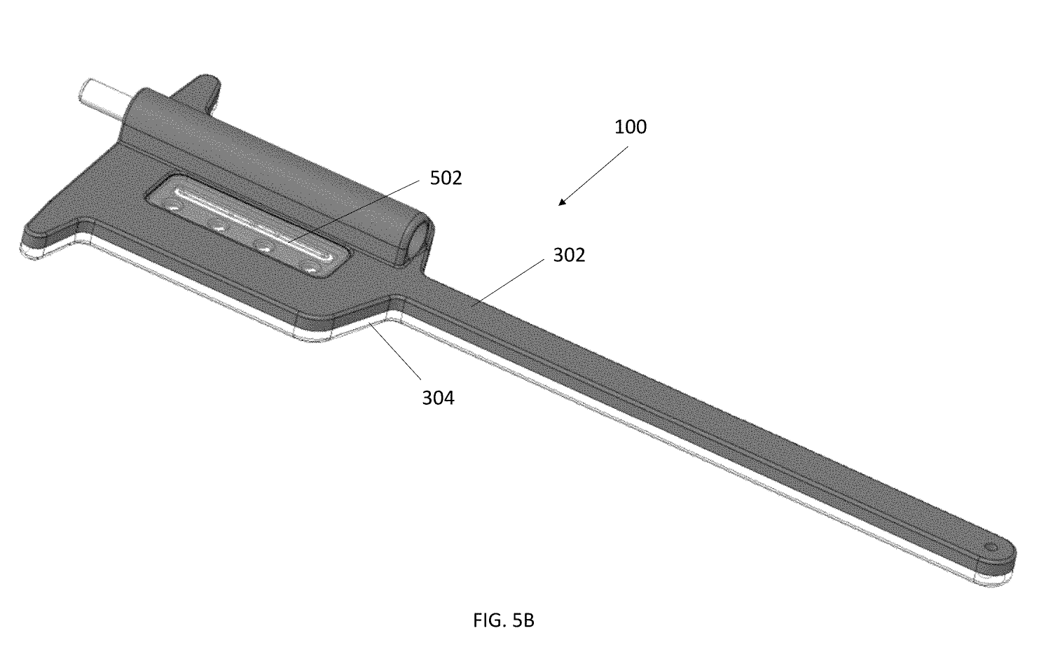

In suitable embodiments, cartridge 100 can be inserted into reader device 602 (or other reader device described herein), and held by mounting apparatus 400, for example as shown in FIG. 4A (420 represents the inside of reader device 602, 422 represents the outside of reader device 602). Mounting apparatus 400 can include various components, such as base 402, lower heater 404, upper heater 406, and mount 408. Mount 408 can be various movable sections, kinematic mounts, ramps (e.g., ramp/support platform 410), pins, springs or pistons that allow for cartridge 100 to be inserted, while allowing for control of movement in 6 degrees of freedom and then held in place for analysis and measurement. For example, ball-tipped screws can be used as mounts 408, and cone, grove or flat features molded or otherwise provided on the surface of cartridge 100 such that cartridge moves in a precise and repeatable positioning when inserted into reader device 602.

An alternative embodiment is shown in FIGS. 4B-4F, illustrating a further mounting apparatus 450 which can be used in the reader device of FIG. 6A or 6C, or as otherwise described herein. FIGS. 4B-4D show cut-away views of mounting apparatus 450, with additional components added in each Figure. Cartridge 100 can be lifted onto mounting pins, e.g., mounting pin 452, via a ramp 454 (see FIG. 4B) or sliding mechanism, such that the mounting pin(s) 452 then pass through alignment holes in the cartridge (not shown) to hold the cartridge in place. This repeatable positioning is very important to ensure accurate readings via the internal components of reader device 602, including various optical components and light sources, as well as readers (e.g., photodiodes). As shown in FIG. 4C, mounting apparatus 450 can include a spring plate 456, such that following the insertion of cartridge 100 into mounting apparatus 450, spring plate 456 can hold cartridge in place. Spring plate 456 can also further include a light shield 458, to cover the positions of sample wells 104. Mounting apparatus 450 also suitably further includes a cover 460, which can function as a heating apparatus, if desired, and hold the remaining components of mounting apparatus 450 together.

FIG. 4E shows a cross section of mounting apparatus 450 and the position of cartridge 100, prior to sliding up ramp 454, and prior to engaging with or seating on mounting pins 452. Portions of light shield 458 of spring plate 456, can be seen extending above the top of mounting apparatus, as spring plate 456 moves upward to allows for the insertion of cartridge 100. Upon reaching the end of ramp 454, cartridge drops down/engages onto mounting pins 452, to hold cartridge in place 100 (see FIG. 4F). Spring plate 456 also drops down to provide further stability to cartridge 100 in mounting apparatus 450. Mounting pins 452 can no longer be seen, as they are engaged with alignment holes in the cartridge.

As shown, mounting apparatus 400 suitably includes lower heater 404 and upper heater 406 which provide heating to the optical sample wells and heat sample to an appropriate temperature to allow the enzymatic reactions described herein to take place. Generally, the temperature for optimizing the reaction between the sample and the hemocyte lysate is about 25° C. to about 40° C., or about 30° C. to about 40° C. The heating time of the sample is generally on the order of about 10-30 minutes to allow for the reaction to occur and the optical property of the sample to be measured and recorded.

Mounting apparatus, including mounts 408, can also be used to shake or mix the sample in optical sample well 104, prior to measuring an optical property of the sample. Fluid flow, including turbulent flow, can be used to encourage mixing of the sample in optical sample well 104. In additional embodiments, a sonicator, vacuum device, or other device can also be used to provide agitation to the sample to aid in mixing.

FIG. 6B shows an illustration of cartridge 100 inserted into reader device 602, such that the measurement of an optical property of the sample can take place. As shown, reader device 602 is suitably a hand-held or readily portable device that can be taken into various laboratory or clinical settings and easily manipulated and controlled via simple touch screen commands.

FIG. 6C shows an illustration of cartridge 100 inserted into an additional reader device 604, such that the measurement of an optical property of the sample can take place. As shown, additional reader device 604 is suitably a hand-held or readily portable device that can be taken into various laboratory or clinical settings and easily manipulated and controlled via simple touch screen commands. Additional reader device 604 can accommodate the insertion of the full length of cartridge 100, thereby containing the cartridge completely within the reader device, prior to measurement. Dimensions are exemplary and shown in millimeters. As indicated, insertion of cartridge 100 is suitably carried out such that the section of the cartridge containing the sample wells is inserted first, and thus fully contained within the device. This orientation of insertion also helps with moving the two position syringe into the second position to provide transport from conduit 108 to optical sample wells 104.

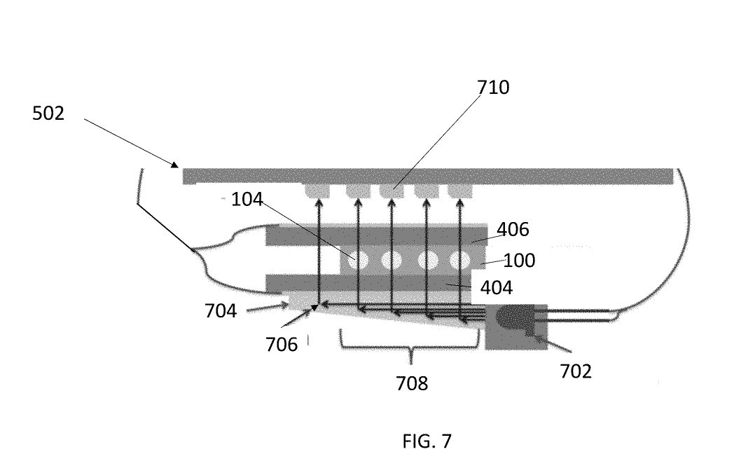

FIG. 7 shows exemplary components of reader device 602 (or additional reader device 604) which can be used to take a measurement of an optical property of a sample. In embodiments, reader device 602/604 can include light source 702, suitably an LED light source which is capable of producing light at a wavelength of about 350 nm to about 450 nm, or about 400 nm to about 410 nm, or about 405 nm. Also included in reader device 602/604 is light panel 704 (for example a light guide, mirror, prism, etc.), which provides illumination to each of optical sample wells 104, to provide signal channels 708, which can be read by detector 710, suitably an array of photodiodes. A further reference channel 706 is provided as a control to determine if the correct intensity and wavelength of light is being provided. As shown, cartridge 100 is positioned between upper heater 406 and lower heater 404, which provides heating to cartridge 100, suitably to about 25° C. to about 40° C., to facilitate the enzymatic reaction. Exemplary heaters can be prepared from flexible films of polyimide, for example DUPONT KAPTON®, and silicone rubber. The orientation of signal channels 708 and detector 710 is dictated by the orientation of sample wells 104 in cartridge 100.

Various other components of reader device 602/604, such as computer circuitry for conducting the determination and/or quantification of the amount of absorbance are well known in the art and can be readily integrated into such devices.

Upon insertion of cartridge 100 into reader device 602/604, heating can occur via upper heater 406 and lower heater 404, to facilitate the desired enzymatic reaction. Light from light source 702 is provided through optical sample wells 104, which contain the sample, and the absorbance is read at detector 710, suitably a photodiode. The absorbance from the various optical sample wells are then compared, with suitably one or more of the optical sample wells acting as a control which contains a microbial contaminant. The presence and/or amount of endotoxin in the sample can then be determined by comparing the amount in the sample, with that in the control, to a standard calibration curve, for example. An ordinarily skilled artisan can readily prepare such standard calibration curves using absorbance of known amounts of endotoxin. In additional embodiments, reader device 602/604 can also be provided with archived (i.e., maintained and initially provided on the reader device 602 or additional reader device 604), or pre-determined standard curves, that can be used by an operator to determine the amount of endotoxin in a test sample. Such archived or pre-determined standard curves can be provided for various endotoxins and can be updated as needed by a user, for example by downloading standard curves from a maintenance database, etc. As shown in FIGS. 6A-6C, multiple cartridges 100 can be inserted into reader device 602 or additional reader device 604 and read at the same time, allowing for the presence and/or quantity of microbial contaminants in many different samples to be determined at the same time.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims or items are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A cartridge for determining the presence and/or amount of a microbial contaminant in a sample, the cartridge comprising:
a. a housing including an optical sample well, a fluid inlet port and a conduit fluidly connecting the fluid inlet port and the optical sample well;
b. a pump mechanism associated with the housing and fluidly connected to the fluid inlet port and the conduit; and
c. a dried composition including a hemocyte lysate dried on the optical sample well.

2. The cartridge of claim 1, wherein the housing includes four optical sample wells, each comprising the dried composition including the hemocyte lysate and further including a chromogenic substrate dried on the optical sample wells, and wherein two of the four optical sample wells further include an agent representative of the microbial contaminant dried on the optical sample wells.

3. The cartridge of claim 1, wherein the housing includes a liquid impermeable membrane fluidly connected to the optical sample well.

4. The cartridge of claim 1, wherein the housing includes a top section and a bottom section, mechanically connected to each other.

5. The cartridge of claim 1, wherein the pump mechanism is a two-position syringe, wherein a first position creates a vacuum to introduce the sample into the fluid inlet port and the conduit, and a second position provides transport from the conduit to the optical sample well.

6. The cartridge of claim 1, wherein the hemocyte lysate is limulus amoebocyte lysate.

7. The cartridge of claim 2, wherein the agent representative of the microbial contaminant is a bacterial endotoxin.

8. A cartridge for determining the presence and/or amount of a microbial contaminant in a sample, the cartridge comprising:
a. a housing including a first optical sample well and a second optical sample well, a fluid inlet port and a conduit fluidly connecting the fluid inlet port and the first optical sample well and the second optical sample well;
b. a two-position syringe attached to the housing and fluidly connected to the fluid inlet port and the conduit, wherein a first position creates a vacuum to introduce the sample into the fluid inlet port and the conduit, and a second position provides transport from the conduit to the first optical sample well and the second optical sample well;
c. a dried composition including a hemocyte lysate and a chromogenic substrate dried on each of the first optical sample well and the second optical sample well; and
d. an agent representative of the microbial contaminant dried on the second optical sample well.

9. The cartridge of claim 8, wherein the housing includes four optical sample wells, each comprising the dried composition dried on the optical sample wells, and wherein two of the four optical sample wells include the agent representative of the microbial contaminant dried on the optical sample wells.

10. The cartridge of claim 8, wherein the housing includes a liquid impermeable membrane fluidly connected to the optical sample wells.

11. The cartridge of claim 8, wherein the housing includes a top and a bottom section, mechanically connected to each other.

12. The cartridge of claim 8, wherein the hemocyte lysate is limulus amoebocyte lysate.

13. The cartridge of claim 8, wherein the agent is a bacterial endotoxin.

14. A method for detecting the presence of a microbial contaminant in a sample, the method comprising:
a. introducing the sample into the fluid inlet port of the cartridge of claim 1 and transferring the sample to the conduit;
b. transferring the sample from the conduit to the optical sample well; and
c. measuring an optical property of the sample in the optical sample well, wherein a change in the optical property is indicative of the presence of the microbial contaminant in the sample.

15. The method of claim 14, wherein the measuring the optical property is a change in absorbance of light at a preselected wavelength.

16. The method of claim 15, wherein the change in absorbance of light at a preselected wavelength is compared to a standard curve.

17. The method of claim 16, wherein the standard curve is an archived standard curve.

18. The method of claim 14, wherein the introducing the sample comprises creating a vacuum via the pump mechanism to introduce the sample into the fluid inlet port and the conduit.

19. The method of claim 14, wherein the transferring comprises transporting via the pump mechanism the sample from conduit to the optical sample well, and wherein the transporting is stopped by a liquid impermeable membrane fluidly connected to the optical sample well.

20. A method for detecting the presence of a microbial contaminant in a sample, the method comprising:
a. introducing the sample into the fluid inlet port of the cartridge of claim 8 and transferring the sample to the conduit;
b. transferring the sample from the conduit to the optical sample wells; and
c. measuring an optical property of the sample in the optical sample wells, wherein a change in the optical property is indicative of the presence of the microbial contaminant in the sample.

21. The method of claim 20, wherein the measuring the optical property is a change in absorbance of light at a preselected wavelength.

22. The method of claim 21, wherein the change in absorbance of light at a preselected wavelength is compared to a standard curve.

23. The method of claim 22, wherein the standard curve is an archived standard curve.

24. The method of claim 20, wherein the introducing the sample comprises creating a vacuum via the two-position syringe to introduce the sample into the fluid inlet port and the conduit.

25. The method of claim 18, wherein the transferring comprises transporting via the two-position syringe the sample from the conduit to the optical sample wells, and wherein the transporting is stopped by a liquid impermeable membrane fluidly connected to the optical sample wells such that final volumes of the samples in each of the sample wells varies by less than about 10%.
